(19) United States
(12) Patent Application Publication
(10) Pub. No.: US 2025/0049869 A1
(43) Pub. Date: Feb. 13, 2025

(54) USE OF AURICULARIA AURICULA TOTAL POLYSACCHARIDE OR MONOMERIC POLYSACCHARIDE IN PREPARATION OF PD-L1 INHIBITOR

(71) Applicant: Yonggang XIA, Harbin (CN)

(72) Inventors: Yonggang XIA, Harbin (CN); Jun LIANG, Harbin (CN)

(73) Assignee: Yonggang XIA, Harbin (CN)

(21) Appl. No.: 18/900,976

(22) Filed: Sep. 30, 2024

(30) Foreign Application Priority Data
Aug. 2, 2023 (CN) 202310966431.2

(51) Int. Cl. A61K 36/07; A61K 31/736; A61P 35/00

(52) U.S. Cl. CPC A61K 36/07; A61K 31/736; A61P 35/00; A61K 2236/331; A61K 2236/53; A61K 2236/55

(57) ABSTRACT

A use of Auricularia auricula total polysaccharide or monomeric polysaccharide in preparation of a PD-L1 inhibitor is provided. The Auricularia auricula total polysaccharide or monomeric polysaccharide both contain acetyl active groups. The Auricularia auricula total polysaccharide or monomeric polysaccharide containing acetyl active groups can effectively inhibit the expression of PD-L1 protein in tumor cells, inhibit the proliferation of tumor cells, and can be used as a natural PD-L1 inhibitor for the treatment of various tumors with high expression of PD-L1, such as lung cancer and colon cancer. Compared with the existing PD-L1 inhibitors, the use of Auricularia auricula total polysaccharide or monomeric polysaccharide as a PD-L1 inhibitor has the advantages of non-toxic side effects, low price, and wide adaptation to patient groups.

### CROSS REFERENCE TO THE RELATED APPLICATIONS

This application is based upon and claims priority to Chinese Patent Application No. 202310966431.2, filed on Aug. 2, 2023, the entire contents of which are incorporated herein by reference.

### TECHNICAL FIELD

The present disclosure relates to a new pharmacological use of Auricularia auricula total polysaccharide and monomeric polysaccharide, in particular to a use of Auricularia auricula total polysaccharide and monomeric polysaccharide in the preparation of PD-L1 inhibitors, belonging to the new application field of Auricularia auricula total polysaccharide and monomeric polysaccharide.

### BACKGROUND

Programmed cell death ligand 1 (PD-L1) inhibitors, as immune activating drugs, can block the interaction between programmed cell death protein 1 (PD-1) and PD-L1, which is an effective means to activate tumor immune response, and can achieve ideal anti-tumor effects without affecting the normal immune function of body cells. PD-L1 inhibitors are currently on the market, and the drugs currently approved for indications include Durvalumab, Atezolizumab, and Sugemalimab. Durvalumab is mainly used in stage III non-small cell lung cancer, Atezolizumab is mainly used in extensive-stage small cell lung cancer, and Sugemalimab is mainly used in advanced non-small cell lung cancer. Adebrelimab is a self-developed PD-L1 inhibitor drug in China. Clinical studies have confirmed that this immune drug combined with chemotherapy can significantly prolong the survival time of patients with extensive-stage small cell lung cancer compared with traditional chemotherapy. However, PD-L1 inhibitors are known to have a high incidence of adverse reactions and are expensive. Therefore, there is an urgent need to develop a PD-L1 inhibitor that reduces or eliminates side effects, is cheap to produce, and is suitable for most clinical patients, so as to fundamentally improve the clinical efficacy of PD-L1 inhibitors in cancer patients.

The scientific name Auricularia auricula-judae. belongs to the Auriculariaceae family. Auricularia auricula is commonly found in dried fruiting bodies of agarics. According to the theory of traditional Chinese medicine, the nature and flavour of Auricularia auricula are neutral and sweet, and it has the functions of nourishing yin and tonifying blood, improving eyesight, clearing the lungs, etc., which are recorded in many ancient books. Auricularia auricula, which is hardy and mainly abundant in northern China, is one of the precious local fungus resources in Heilongjiang Province. So far, no literature has suggested the potential of Auricularia auricula polysaccharides as PD-L1 inhibitors.

### SUMMARY

The first purpose of the present disclosure is to provide a use of Auricularia auricula total polysaccharide in preparation of PD-L1 inhibitors or oncology drugs;

The second purpose of the present disclosure is to provide a use of the monomeric polysaccharide isolated from Auricularia auricula in preparation of PD-L1 inhibitors or oncology drugs;

The above purposes of the present disclosure are realized through the following technical schemes:

On the one hand, the present disclosure provides the use of Auricularia auricula total polysaccharide in preparation of PD-L1 inhibitors, where the Auricularia auricula total polysaccharide contains acetyl active groups. The Auricularia auricula total polysaccharide containing acetyl active groups obtained from Auricularia auricula by any extraction method disclosed by the prior art or any Auricularia auricula total polysaccharide containing acetyl active groups obtained through commercial means can be applicable to the present disclosure. Preferably, the content of acetyl active groups in Auricularia auricula total polysaccharide is preferably more than 10%, preferably 15-30%.

As a reference, the present disclosure provides a preparation method of Auricularia auricula total polysaccharide containing acetyl active groups, including:
(1) extracting Auricularia auricula with water as extraction solvent to obtain Auricularia auricula water extract; and
(2) removing the impurities in the Auricularia auricula water extract to obtain Auricularia auricula total polysaccharide containing acetyl active groups.

As a preferred embodiment, in step (1), water was used as an extraction solvent to extract Auricularia auricula water extract from Auricularia auricula by boiling water extraction method. The boiling water extraction method includes, but is not limited to, the decocting method or reflux extraction method, etc.

As a preferred embodiment, the impurities described in step (2) include small-molecule compounds, oligosaccharides, or soluble salts. The method for removing the impurities in the Auricularia auricula water extract includes the interception treatment of the Auricularia auricula water extract by using an ultrafiltration chromatographic column. The interception treatment range of the ultrafiltration chromatographic column is selected from 3K, 5K, 10K, or 50K, and 50K is the best choice; and in the interception treatment, according to mL/g, the ratio of water to Auricularia auricula water extract is 7:1, and the ultrafiltration pressure is 2 Mpa.

As a preferred embodiment, the present disclosure provides Auricularia auricula total polysaccharide containing acetyl active groups prepared by the above preparation method, whose molecular weight distribution includes 1737 KDa, 308.5 KDa, and 145.4 KDa; according to the molar percentage of monosaccharide composition, xylose:glucuronicacid:galactose:glucose:mannose=25:7:2:3:63; the content of acetyl groups was 20.47%; the total sugar content was 75.09±5% (of which the content of uronic acid was 14.53±2%) and the protein content was 25.45±5%.

Western blot (WB) showed that Auricularia auricula total polysaccharide METP inhibited the expression of PD-L1 protein in H157 cells. However, the inhibitory effect of deacetylated monomeric polysaccharide (DME-2) on PD-L1 protein in H157 cells was not obvious, and the inhibitory effect on PD-L1 protein in H157 cells was significantly weaker than that of ME-2. The experiment proved that the Auricularia auricula total polysaccharide containing acetyl active groups can directly down-regulate the PD-L1 expression on the surface of NCI-H157 cells, and the inhibition rate of PD-L1 reached 51.4%, which can be used for the treatment of various cancers with high expression of PD-L1, such as lung cancer and colon cancer.

On the other hand, the present disclosure provides the use of monomeric polysaccharide isolated from Auricularia auricula in preparation of PD-L1 inhibitors. The monomeric polysaccharide isolated from Auricularia auricula is the monomeric polysaccharide isolated from Auricularia auricula disclosed in the invention patent application No. CN 114957508A, and the content of the acetyl active groups of the monomeric polysaccharide is 18.0%.

The monomeric polysaccharide is a high-acetyl glucuronoxylogalactoglucomannan, which is composed of xylose, glucuronic acid, galactose, glucose, and mannose, where the theoretical composition of xylose:glucuronicacid:galactose:glucose:mannose has a molar ratio of 3:4:1:1:11.

Further, the sugar residue linking composition of the monomeric polysaccharide is as follows: →3)-Manp-(1→, →2,3)-Manp-(1→, →2,3,6)-Manp-(1→, →3,6)-Manp-(1→, Manp-(1→, Glcp-(1→, GlcAp-(1→, Xylp-(1→, and Galp-(1→; and the →3)-Manp-(1→, →2,3)-Manp-(1→, →2,3,6)-Manp-(1→, →3,6)-Manp-(1→, Manp-(1→, Glcp-(1→, GlcAp-(1→, Xylp-(1→, and Galp-(1→; the theoretical ratio was 3:1:3:3:1:1:4:3:1.

Further, the chemical fine structure repeating unit of the monomeric polysaccharide is shown as follows:

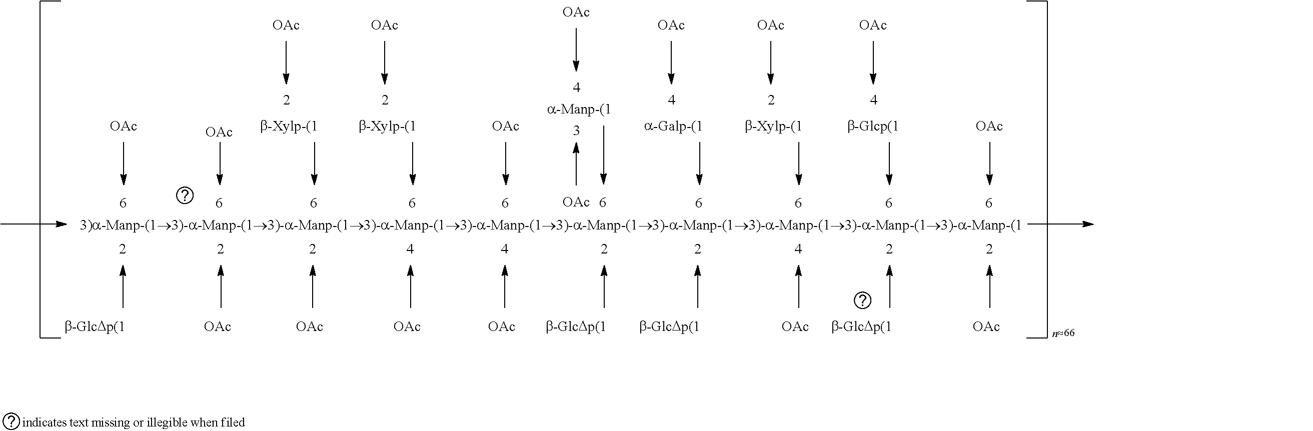

In order to verify whether the monomeric polysaccharide ME-2 isolated from Auricularia auricula has an inhibitory effect on the expression and function of PD-L1 protein after binding with PD-L1 protein, that is, whether the Auricularia auricula total polysaccharide with acetyl group content greater than 10% or the Auricularia auricula monomeric polysaccharide ME-2 can be used as a PD-L1 inhibitor. The present disclosure first detected the PD-L1 expression level of seven different tumor cells, including non-small cell lung cancer NCI-H157, A549, and LLC, human osteosarcoma U2OS, colon cancer SW620, liver cancer HepG-2, and breast cancer MCF-7, by Western blot, and then performed cytotoxicity tests by the cell counting kit-8 (CCK8) method. The results showed that the proliferation of non-small cell lung cancer NCI-H157 and mouse lung cancer LLC cells was not significantly inhibited, and the monomeric polysaccharide ME-2 had no obvious cytotoxicity to NCI-H157 and LLC cells, indicating that Auricularia auricula monomeric polysaccharide ME-2 itself was not cytotoxic and did not inhibit the proliferation of tumor cells through cytotoxic effects.

Further studies showed that the Auricularia auricula monomeric polysaccharide ME-2 down-regulated PD-L1 in all four types of tumor cells with high expression of PD-L1, and the down-regulated trend was most obvious in non-small cell lung cancer H157 and mouse lung cancer LLC cells. The down-regulation of PD-L1 on tumor cell surface was concentration- and time-dependent. The down-regulation of PD-L1 was most significant when the dose concentration was 2 mg/mL and the administration time was 24 h.

The present disclosure used interferon IFN-γ to induce high expression of PD-L1 in non-small cell lung cancer A549, liver cancer HepG-2, and breast cancer MCF-7 cells, and acted Auricularia auricula monomeric polysaccharide ME-2 and IFN-γ together on cells. The experimental results showed that Auricularia auricula monomeric polysaccharide ME-2 can significantly down-regulate the IFN-γ-induced high expression of PD-L1, which proved that Auricularia auricula monomeric polysaccharide ME-2 can significantly down-regulate the expression of PD-L1 in both PD-L1 high-expression and induced PD-L1 high-expression tumor cells.

The present disclosure adopted flow cytometry to detect the effect of Auricularia auricula monomeric polysaccharide ME-2 on the abundance of expression of PD-L15 in lung cancer cells. The results showed that the fluorescence intensity of the two kinds of lung cancer cells gradually decreased with the increase in concentration, which further verified that Auricularia auricula monomeric polysaccharide ME-2 can down-regulate the expression of PD-L1 in non-small cell lung cancer and metastatic lung cancer cell lines.

On this basis, the present disclosure further detected whether the down-regulation of PD-L1 mediated by Auricularia auricula monomeric polysaccharide ME-2 can affect the binding level of PD-L1 in tumor cells and PD-1 protein in T cells. The results showed that the interaction of PD-1/PD-L1 was blocked by Auricularia auricula monomeric polysaccharide ME-2 in a dose-dependent manner. Further, the killing effect of Jurkat T cells mediated by Auricularia auricula monomeric polysaccharide ME-2 on lung cancer cells was verified by co-culture of tumor cells and T cells. The results showed that ME-2-mediated Jurkat T cells block the PD-L1/PD-1 interaction, playing a role in tumor killing, thus contributing to the recovery of the biological function of immune checkpoints in the body.

The present disclosure also constructs a lung cancer transplant tumor model to treat solid tumors in mice by oral administration of Auricularia auricula monomeric polysaccharide ME-2. According to the experimental results, ME-2 measured at 100 mg/kg can significantly inhibit the proliferation of tumors in mice, and the effect was obvious better than a positive drug: the tumor morphology changed obviously, and the tumor volume gradually became smaller or even disappeared. Body weight, serum biochemical indexes, and pathological sections of vital organs of mice were monitored. The experimental results all proved that Auricularia auricula monomeric polysaccharide ME-2 measured at 100 mg/kg had no obvious toxic and side effects on mice, and the quantitative polymerase chain reaction (qPCR) confirmed that Auricularia auricula monomeric polysaccharide ME-2 could significantly inhibit the expression of PD-L1 mRNA in solid tumors.

In conclusion, the Auricularia auricula total polysaccharide METP and monomeric polysaccharide ME-2 can be used as natural PD-L1 inhibitors for the treatment of various cancers with high expression of PD-L1, such as lung cancer and colon cancer, and have wide application value. The acetyl group is an important active group of Auricularia auricula total polysaccharide METP and monomeric polysaccharide ME-2 as PD-L1 inhibitors. Compared with the existing PD-L1 inhibitors, METP and ME-2 have the advantages of non-toxic side effects, low price, and wide adaptation to patient groups.

Therefore, on the other hand, the present disclosure provides a pharmaceutical composition for inhibiting PD-L1, which is composed of an effective amount of Auricularia auricula total polysaccharide or Auricularia auricula monomeric polysaccharide and an acceptable excipient or carrier in a pharmaceutical preparation, where the Auricularia auricula total polysaccharide or Auricularia auricula monomeric polysaccharide contains acetyl active groups.

On the other hand, the present disclosure provides a pharmaceutical composition for treating tumors, which is composed of an effective amount of Auricularia auricula total polysaccharide or Auricularia auricula monomeric polysaccharide and an acceptable excipient or carrier in a pharmaceutical preparation, where the Auricularia auricula total polysaccharide or the Auricularia auricula monomeric polysaccharide contains acetyl active groups, and the tumors include non-small cell lung cancer, osteosarcoma, colon cancer, liver cancer, breast cancer, etc., but are not limited to the above tumor types.

Those skilled in the art may prepare the pharmaceutical composition into a conventional pharmaceutical preparation according to conventional methods in the field of pharmaceutical preparations; the dosage form of the pharmaceutical preparation may be in solid, semi-solid, or liquid form; preferably freeze-dried powder, tablet, capsule, soft capsule, granule, pill, oral liquid, dry suspension, dropping pill, dry extract, injection, or infusion.

The pharmaceutical preparation is administered orally or by injection.

The excipients or carriers in the present disclosure refer to conventional excipients or carriers in the pharmaceutical field, such as diluent, disintegrant, lubricant, excipient, binder, flow aid, filler, surfactant, etc.; in addition, other adjuvants, such as fragrance and sweetener, can be added to the composition. The diluent may be one or more components that increase the weight and volume of the tablet. Commonly used diluents include lactose, starch, pregelatinized starch, microcrystalline cellulose, sorbitol, mannitol, and inorganic calcium salt. The most commonly used are lactose, starch, and microcrystalline cellulose. The disintegrant may be one or several mixtures of cross-linked polyethylpyrrolidone (2-6% ratio to total weight), cross-linked sodium carboxymethyl cellulose (2-6% ratio to total weight), alginate (2-5% ratio to total weight), and microcrystalline cellulose (5-15% ratio to total weight). The lubricant includes one or several mixtures of stearic acid, sodium stearate, magnesium stearate, calcium stearate, polyethylene glycol, talcum powder, and hydrogenated vegetable oil. The dosage range of the lubricant (ratio to total weight) is 0.10-1%, and the general dosage is 0.25-0.75%. The binder may be one or more components conducive to granulation; it can be starch paste (10-30%, ratio to the total weight of the binder), hydroxypropyl methyl cellulose (2-5%, ratio to the total weight of the binder), polyvinylpyrrolidone (2-20%, ratio to the total weight of the binder), and an ethanol solution of polyvinylpyrrolidone is preferred. The flow aid can be one or several mixtures of colloidal silicon dioxide, talcum powder, and magnesium trisilicate. The surfactant may be one or more components capable of improving wettability and increasing drug dissolution, commonly sodium lauryl sulfate (common range is 0.2-6%, ratio to the total weight).

### BRIEF DESCRIPTION OF THE DRAWINGS

FIGS. 1A-1G show the chemical composition of Auricularia auricula total polysaccharides (METP); (FIG. 1A) multi-angle laser scattering diagram of Auricularia auricula total polysaccharide METP; (FIG. 1B) chromatogram of monosaccharide composition of Auricularia auricula total polysaccharide by high-performance anion-exchange chromatography with pulsed amperometric detection (HPAEC-PAD); (FIG. 1C) chromatogram of acetyl group content of Auricularia auricula total polysaccharide METP; (FIG. 1D) standard curve of total sugar content of Auricularia auricula total polysaccharide METP; (FIG. 1E) standard curve of uronic acid content of Auricularia auricula total polysaccharide METP; (FIG. 1F) standard curve of protein content of Auricularia auricula total polysaccharide METP; (FIG. 1G) chromatogram of acetyl group content of Auricularia auricula monomeric polysaccharide ME-2.

FIGS. 2A-2E show the targeted inhibition of ME-2 on the specific binding of PD-L1 and PD-1 evaluated by surface plasmon resonance (SPR) method.

FIGS. 3A-3B. show the effect of Auricularia auricula monomeric polysaccharide ME-2 on the expression level of PD-L1 on the surface of seven different tumor cells by Western blot.

FIGS. 4A-4B show the cell proliferation changes of lung cancer NCI-H157 (FIG. 4A) and LLC (FIG. 4B) cells after 24 h treatment with different concentrations of Auricularia auricula monomeric polysaccharide ME-2 detected by the CCK8 method. The experimental results were statistically analyzed, with *P<0.05; **P<0.01; ***P<0.001.

FIGS. 5A-5B show the effect of Auricularia auricula monomeric polysaccharide ME-2 on the expression level of PD-L1 in tumor cells detected by Western blot with gradient concentration and time; (FIG. 5A) the effect of different concentrations of ME-2 on PD-L1 expression on the surface of NCI-H157, LLC, U20S, and SW620 cancer cells; (FIG. 5B) the effect of ME-2 on PD-L1 expression on the surface of NCI-H157, LLC, U20S, and SW620 cancer cells under different action time. The experimental results were statistically analyzed, with *P<0.05; **P<0.01; ***P<0.001.

FIG. 6 shows the effect of Auricularia auricula monomeric polysaccharide ME-2 on PD-L1 expression in IFN-γ-induced tumor cells with high expression of PD-L1. The experimental results were statistically analyzed, with *P<0.05; **P<0.01; ***P<0.001.

FIG. 7 shows the effect of Auricularia auricula monomeric polysaccharide ME-2 on PD-L1 expression in lung cancer cells verified by flow cytometry. The experimental results were statistically analyzed, with *P<0.05; **P<0.01; ***P<0.001.

FIGS. 8A-8B show the blocking effect of Auricularia auricula monomeric polysaccharide ME-2 on PD-L1/PD-1 axis detected by flow cytometry (FIG. 8A) and laser confocal method (FIG. 8B). The experimental results were statistically analyzed, with *P<0.05; **P<0.01; ***P<0.001.

FIG. 9 shows the killing effect of Jurkat T cells mediated by Auricularia auricula monomeric polysaccharide ME-2 on lung cancer cells. The experimental results were statistically analyzed, with *P<0.05; **P<0.01; ***P<0.001.

FIGS. 10A-10H show the anti-tumor effect and biosafety verification of Auricularia auricula monomeric polysaccharide ME-2 on tumor-bearing mice; (FIG. 10A) experimental design of mouse tumor transplantation; (FIG. 10B) morphology and size of mouse transplanted tumor; (FIG. 10C) gravimetric analysis of mouse transplanted tumor; (FIG. 10D) volumetric analysis of mouse transplanted tumor; (FIG. 10E) analysis of PD-L1 mRNA expression levels in mouse transplanted tumor; (FIG. 10F) analysis of body weight changes in mice; (FIG. 10G) pathological changes of mouse organs; (FIG. 10H) changes of blood biochemical indexes of mice. The experimental results were statistically analyzed, with *P<0.05; **P<0.01; ***P<0.001.

FIGS. 11A-11D show the application of Auricularia auricula total polysaccharide METP as a PD-L1 inhibitor; (FIG. 11A) the inhibition levels of ME-2 deacetylation product (dME-2), total polysaccharide METP, and ME-2 on PD-L1 protein in H157 cells were compared by WB methods; (FIGS. 11B-11D) analysis of transplanted tumor volume, transplanted tumor weight, and body weight change in mice. The experimental results were statistically analyzed, among which, *P<0.05; **P<0.01.

### DETAILED DESCRIPTION OF THE EMBODIMENTS

The present disclosure is further described below in conjunction with specific examples; the advantages and characteristics of the present disclosure will become clearer with the description, but these examples are only exemplary and do not constitute any limitation on the scope of the present disclosure. Those skilled in the art should understand that the details and forms of the present disclosure may be modified or replaced without deviating from the spirit and scope of the present disclosure, provided that such modifications and substitutions fall within the scope of protection of the present disclosure.

### Example 1 Preparation of Auricularia auricula Total Polysaccharide and Monomeric Polysaccharide

(1) Preparation of Auricularia auricula Total Polysaccharide METP

About 5 kg of crushed Auricularia auricula was taken; each time 250 g of crushed Auricularia auricula was taken and passed through a 20-mesh sieve; 8 L of distilled water was added each time; and extraction was performed for 3 hours each time. The water extract was filtered through the residue with a 300-mesh silk cloth; the residue was extracted twice, and the Auricularia auricula water extracts were combined. The Auricularia auricula water extract was passed through a 50000 Da molecular weight ultrafiltration chromatography column to further remove small-molecule compounds, oligosaccharides, and soluble salts from the Auricularia auricula water extract and concentrated to 5 L. The Auricularia auricula total polysaccharide was obtained by vacuum freeze-drying, and the extraction rate was 18.5%.

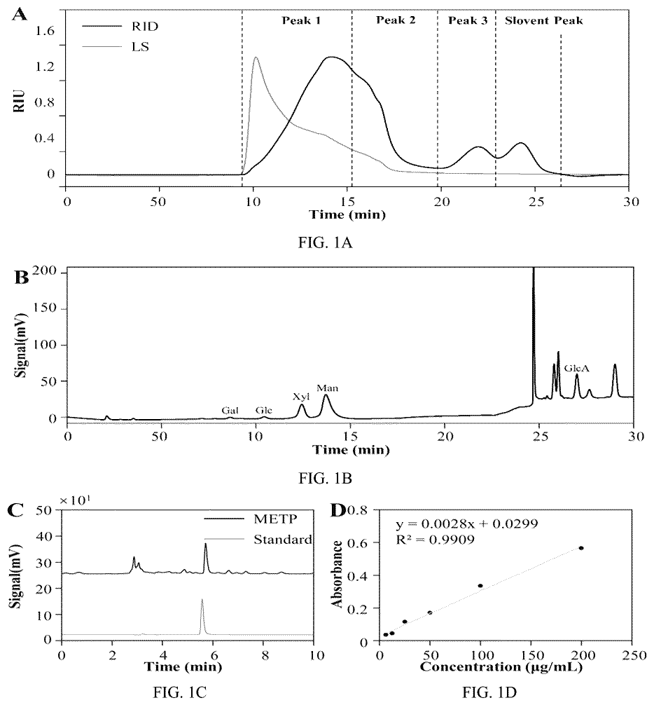

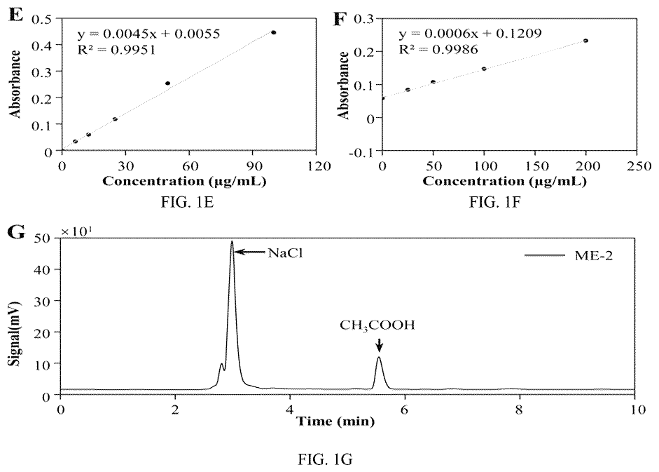

As shown in FIG. 1A, the molecular weight distribution of Auricularia auricula total polysaccharide was determined to include 1737 KDa (Peak 1), 308.5 KDa (Peak 2), and 145.4 KDa (Peak 3) by the multistage hyphenated technique of size exclusion chromatography-multi-angle laser light scattering-refractive index detector (SEC-MALLS-RID). Through the analysis of monosaccharide composition by ion chromatography (HPAEC-PAD) (FIG. 1i), the monosaccharide composition of Auricularia auricula total polysaccharide was determined to be xylose, glucuronic acid, galactose, glucose, and mannose. According to the molar percentage of monosaccharide composition, xylose:glucuronicacid:galactose:glucose:mannose=25:7:2:3:63. By measuring the content of acetic acid, the acetyl group content of Auricularia auricula total polysaccharide was determined by high-performance liquid chromatography (HPLC) as 20.47% (FIG. 1C). By phenol-sulfuric acid method (FIG. 1D), sulfuric acid-carbazole method (FIG. 1E), and coomassie brilliant blue method (FIG. 1F), the total sugar content of Auricularia auricula total polysaccharide was determined to be 70.97%, the uronic acid content was 14.53%, and the protein content was 20.45%. The acetyl group content of Auricularia auricula monomeric polysaccharide ME-2 was determined by HPLC to be 18.0% (FIG. 1G).

SEC-MALLS-RID detection conditions: HPSEC-MALLS-RID was used to determine the molecular weight (M_{w}) of total polysaccharide. HPSEC-MALLS-RID was equipped with Waters-e2695 HPLC (Milford, Massachusetts, USA), Waters-2414 detector (Milford, Massachusetts, USA), and Wyatt DAWN HelEOS-II MALLS Detector (Goleta, California, USA). The chromatographic column was TSK gel G5000PWXL Column (7.8×300 mm, i.d., TOSOH Bioscience, Tokyo, Japan). The mobile phase was 50 mM ammonium formate aqueous solution, and the flow rate was 0.5 mL/min. The Auricularia auricula total polysaccharide (5 mg) was dissolved in 1 mL of mobile phase, centrifuged, and filtered by a 0.22 m aqueous filter membrane.

HPAEC-PAD monosaccharide composition detection conditions: Auricularia auricula total polysaccharide (5 mg) was hydrolyzed with 3 mol/L trifluoroacetic acid (TFA) (3 mL) at 110° C. for 4 h, then washed with methanol for 3 times. The TFA was removed by evaporation and drying. The monosaccharide composition was identified and quantified by HPAEC-PAD (ICS 6000, Thermo Fisher Scientific, USA) after complete acid hydrolysis. The reference electrode is AgCl, and then the sugar was detected with PAD on the gold working electrode. Dionex CarboPac™ PA20 guard column (3×30 mm) and analytical column (3×150 mm, 10 m) were used in the determination. The mobile phase A was water, mobile B was 100 mM NaOH, and mobile C was 100 mM sodium acetate. The flow rate was 0.5 mL/min, the column temperature was 25° C., and the injection volume was 5 L.

HPLC acetyl content detection conditions: Auricularia auricula total polysaccharide (5 mg) was dissolved in 0.20 mol/L NaOH solution and alkaline hydrolyzed at 80° C. for 60 min. 6 mL of anhydrous ethanol was added, followed by shaking violently to mix well, and placing at 4° C. for alcohol precipitation. After centrifugation, the supernatant was transferred to a spinning flask to spin dry. The pH value was adjusted to acidic (pH≤2.2), mixed and diluted to 1 mL with water, and filtered through a 0.22 m microporous filter. The mobile phase A: 0.05% phosphoric acid-water; mobile B: methanol, isocratic elution. The flow rate was 0.8 mL/min, and the detection wavelength was 210 nm.

The detection conditions of the phenol-sulfuric acid method, sulfur-carbazole method, and Coomassie brilliant blue method were as follows: The content of total polysaccharide was determined by the phenol-sulfuric acid method; the standard curve was drawn with mannose as the standard substance, and the curve equation was y=0.0028×+0.0299 (R²=0.9909). The content of uronic acid was determined by the sulfuric acid-carbazole method; the standard curve was drawn with glucuronic acid as the standard substance, and the curve equation was y=0.0045×+0.0055 (R²=0.9951). The protein content was determined by the Coomassie brilliant blue method; the standard curve was drawn with bovine serum albumin as the standard substance, and the curve equation was y=0.0006×+0.1209 (R²=0.9986).

### Experiment Example 1 the Experiment of Auricularia auricula Monomeric Polysaccharide ME-2 as PD-L1 Inhibitor

The Auricularia auricula monomeric polysaccharide ME-2 as a PD-L1 inhibitor is the monomeric polysaccharide isolated from Auricularia auricula disclosed in the Chinese invention patent application with publication No. CN 114957508A.

(1) Affinity Analysis of ME-2 and dME-2 with PD-L1 Protein

Biacore 8K high-throughput molecular interaction analysis system, CM5 chip, and phosphate buffered saline (PBS) buffer as the mobile phase of the instrument were used in the whole process of the surface plasmon resonance experiment. The PD-L1 was coupled to the activated CM5 chip using an amino coupling kit. After two injections of the coupling reagent, the surface coupling strength of the chip barely changed, indicating that the protein coupling was basically completed. The ME-2 polysaccharide solution with different concentrations was passed through the coupling channel and the blank channel at a certain velocity by using the running buffer. After 60 s of dissociation, the chip was regenerated with 10 mM glycine-HCl (pH2.0) and 3 M NaCl at a flow rate of 30 L/min for 30 s. Biacore Evaluation 3.1 software and GraphPad Prism 8.0 software were used to analyze the data and obtain the response curve.

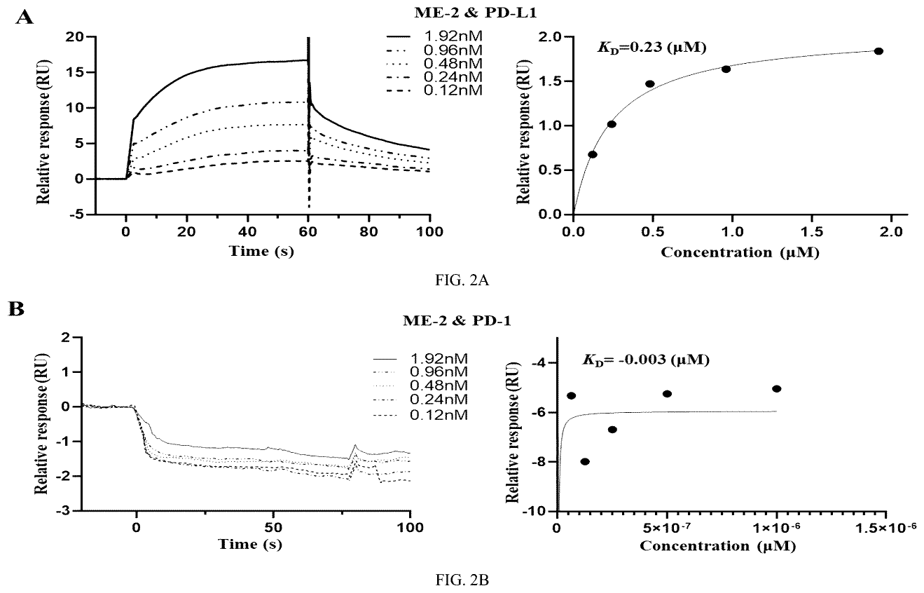

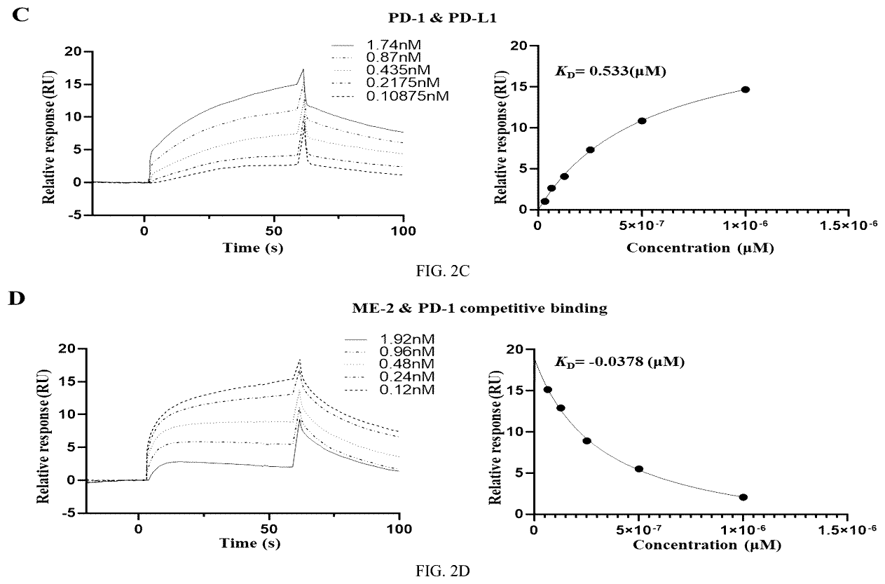

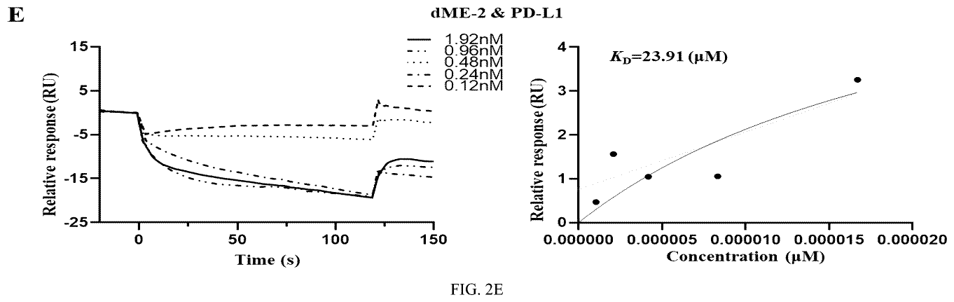

The experimental results are shown in FIGS. 2A-2E. SPR evaluated the affinity between ME-2 and PD-L1 protein, the affinity between ME-2 and PD-1 protein, the affinity between PD-1 and PD-L1 protein, the affinity between PD-L1 and PD-1 competitively inhibited by ME-2, and the affinity between dME-2 and PD-L1 protein, respectively.

The equilibrium dissociation constant was obtained through nonlinear fitting analysis of the data, and it was determined that there was a strong affinity between ME-2 and PD-L1 molecules (FIG. 2A), while ME-2 had no affinity for PD-1 protein (FIG. 2B). As the concentration of ME-2 increased, the response value increased significantly, and the affinity curve was smooth. The regeneration curve decreased obviously, and it had the characteristics of fast bonding and fast dissociation. Its dissociation constant (K_{D}) was 2.323×10⁻⁷, and the K_{D} value of most common small molecule inhibitors of PD-L1 is 10⁻⁶ to 10⁻⁹, indicating that ME-2 showed a strong affinity to PD-L1. However, ME-2 had no affinity for PD-1 protein, indicating that the affinity between ME-2 and PD-L1 had obvious specificity (FIGS. 2A and 2B).

As shown in FIG. 2C, the affinity characteristic between PD-1 and PD-L1 protein was fast binding and slow dissociation, and its affinity constant K_{D} was 5.33×10⁻⁷, which was greater than the K_{D} value of ME-2 and PD-L1, indicating that PD-L1 protein preferentially interacted with ME-2. The competitive experiment between ME-2 and PD-1 further showed that with the increase of the concentration of ME-2, the binding of PD-L1 and PD-1 protein was significantly inhibited, and the affinity curve tended to be flat, while the fitting curve showed a negative trend, which once again proved that ME-2 had a specific affinity for PD-L1 protein. Moreover, ME-2 binding to PD-L1 effectively inhibited the affinity between PD-1 and PD-L1 protein (see FIG. 2D). The KD value of dME-2 and PD-L1 was 2.391×10⁻⁵, and there was no affinity between them (see FIG. 2E), indicating that the acetyl group was an important active group of ME-2.

### (2) Detection of PD-L1 Expression Levels on the Surface of Seven Tumor Cells

In order to further verify whether ME-2 binding with PD-L1 protein had an inhibitory effect on the expression and function of PD-L1 protein, that is, whether high-acetyl ME-2 could be used as a PD-L1 inhibitor, the expression levels of PD-L1 in seven different tumor cells, including non-small cell lung cancer NCI-H157, A549, and LLC, human osteosarcoma U2OS, colon cancer SW620, liver cancer HepG-2, and breast cancer MCF-7, were detected by Western blot.

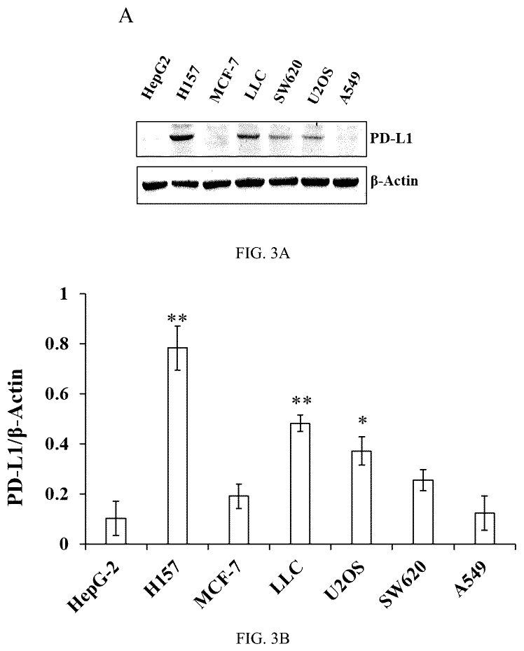

The tumor cells in question were non-small cell lung cancer NCI-H157, mouse lung cancer LLC, colon cancer SW620, human osteosarcoma U2OS, non-small cell lung cancer A549, liver cancer HepG-2, and breast cancer MCF-7. The total protein of the above cells was extracted; the protein concentration was determined by bicinchoninic acid (BCA) assay. The samples were loaded and subjected to sodium dodecyl sulfate (SDS) polyacrylamide gel electrophoresis (SDS-PAGE); the membrane was blocked and immunoreacted after transfer, and an enhanced chemiluminescence (ECL) solution was used for development. Picture processing and gray value analysis were performed on the exposure results on ImageJ software. According to the experimental results, the cell lines with high expression of PD-L1 were selected as follows: NCI-H157, LLC, U2OS, and SW620; and the cell lines with low or no expression of PD-L1 were A549, HepG-2, and MCF-7 (see FIGS. 3A and 3B).

### (3) In Vitro Cytotoxicity Assay

CCK8 was used to detect the cytotoxicity of ME-2 to NCI-H157 and LLC cells. H157 or LLC cells were inoculated overnight in 96-well cell culture plate at a concentration of 1×10⁴/mL. The gradient concentration of ME-2 was prepared in the medium, and CCK8 solution was added into the 96-well cell culture plate after 24 h of action. OD values were measured at 450 nm by an enzyme-linked immunosorbent assay (ELISA) instrument.

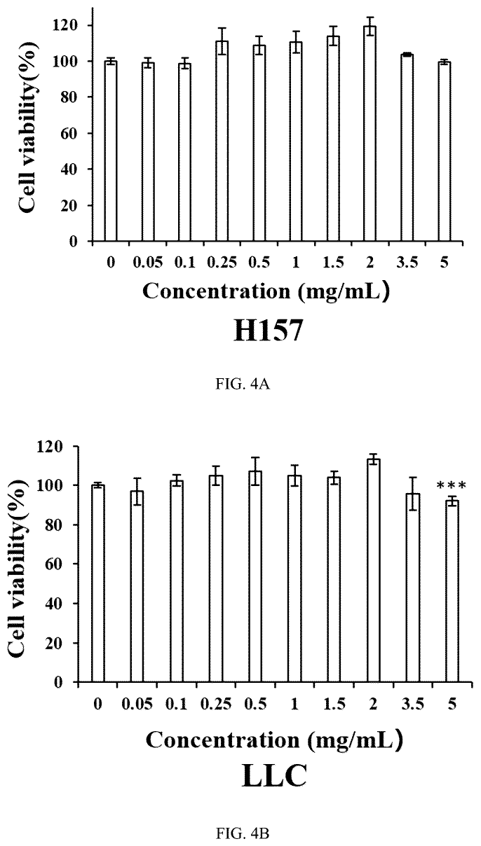

The experimental results showed that with the increase of the concentration gradient of ME-2, the proliferation of NCI-H157 and LLC cells was not significantly inhibited, and ME-2 had no obvious cytotoxicity to NCI-H157 cells (see FIG. 4A) and LLC cells (see FIG. 4B). These results indicate that ME-2 itself is not cytotoxic, and it does not inhibit the proliferation of tumor cells through direct cytotoxic mechanisms.

(4) Effect of Auricularia auricula Monomeric Polysaccharide ME-2 on the Expression Level of PD-L1 in Tumor Cells

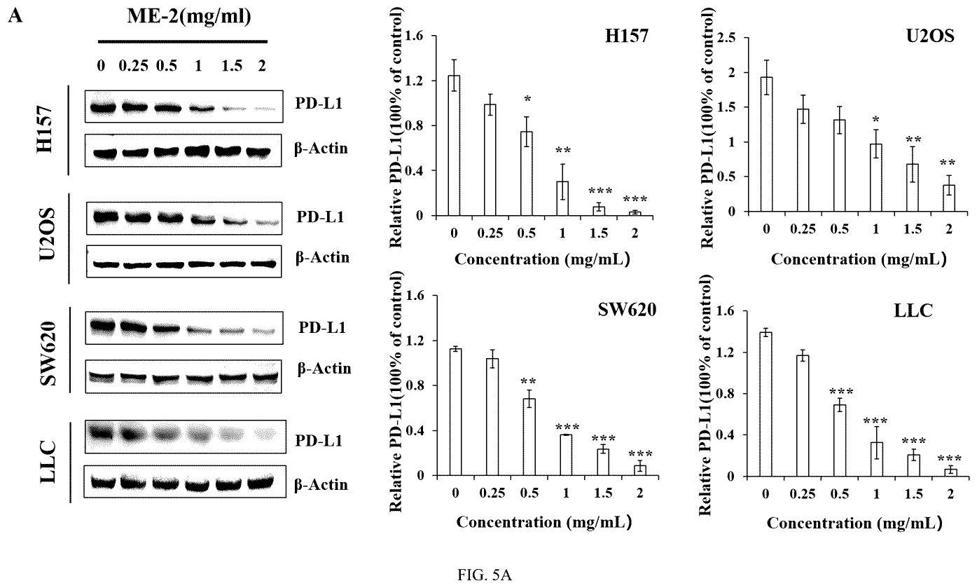

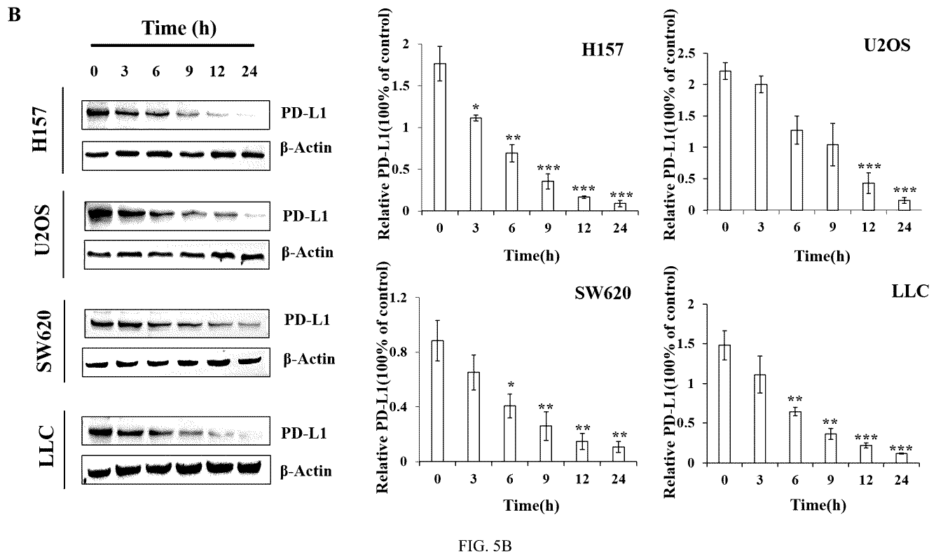

Concentration and time gradient ME-2 were applied to act on four tumor cell lines with high expression of PD-L1, respectively. The Western blot method was used for detection, and the experimental method was shown above. The experimental results showed that ME-2 significantly down-regulated the abundance of PD-L1 in tumor cell lines with high expression of PD-L1, and the down-regulated trend was most obvious in non-small cell lung cancer NCI-H157 and mouse lung cancer LLC cells. The downregulation of tumor cell baseline PD-L1 was concentration dependent (see FIG. 5A) and time dependent (see FIG. 5B). The downregulation of PD-L1 was most significant when the dose concentration was 2 mg/mL and the administration time was 24 h. These results provided the basis for the development and utilization of Auricularia auricula monomeric polysaccharide ME-2 as PD-L1/PD-1 immunosuppressant.

(5) Effect of Auricularia auricula Monomer Polysaccharide ME-2 on IFN-γ-Induced PD-L1 Expression

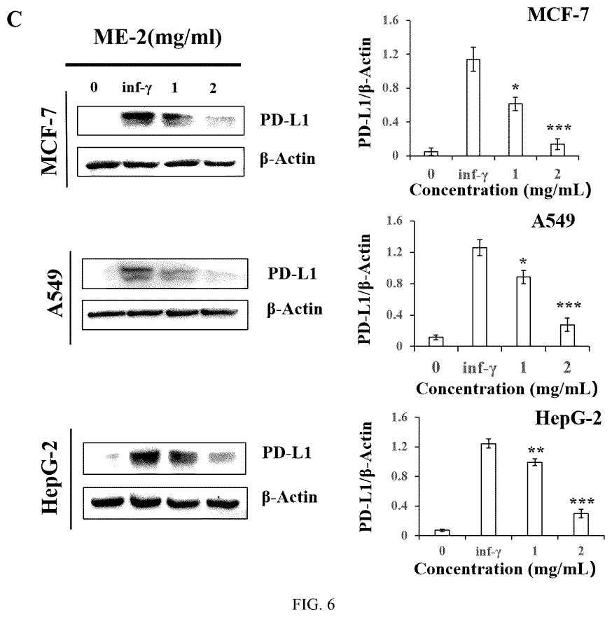

Interferon IFN-γ is an important factor in inducing PD-L1 expression in the tumor immune microenvironment. We used low-expression PD-L1 tumor cells (non-small cell lung cancer A549, liver cancer HepG-2, and breast cancer MCF-7). ME-2 and IFN-γ were co-acted on cells; their expression abundance was detected by the Western blot method, and the experimental method was shown above. The experimental results showed that the Auricularia auricula monomeric polysaccharide ME-2 significantly down-regulated the expression of PD-L1 in IFN-γ-induced A549, HepG-2, and MCF-7 cancer cells with high expression of PD-L1 (see FIG. 6). It was proved that ME-2 could also significantly inhibit the expression of PD-L1 in A549, HepG-2, and MCF-7 cancer cells with high expression of PD-L1 induced by IFN-γ.

(6) Effect of Auricularia auricula Monomeric Polysaccharide ME-2 on PD-L1 Expression in Lung Cancer Cells was Detected by Flow Cytometry

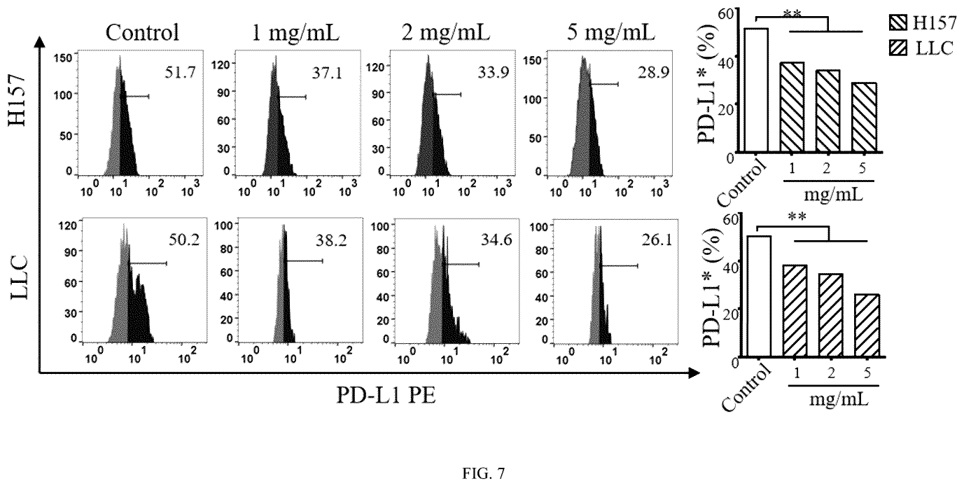

NCI-H157 or LLC cells in a 6-well plate were treated with low, medium, and high doses of ME-2 at 1, 2, and 5 mg/mL, respectively, for corresponding time, and the cells were stained with a fluorescent antibody. Fluorescence intensity was measured by flow cytometry. The experimental results obtained further verified the conclusion obtained in the Western blot above: ME-2 concentration dependent inhibition of PD-L1 expression was visually manifested as a decrease in the fluorescence intensity gradient of PD-L1 in NCI-H157 and LLC lung cancer cells (see FIG. 7), which further verified the feasibility of ME-2 as a PD-L1 inhibitor.

(7) Effect of Auricularia auricula Monomeric Polysaccharide ME-2 on PD-L1/PD-1 Interaction

In order to further verify whether the downregulation of PD-L1 mediated by ME2 affects the binding level of PD-L1 and PD-1 protein in tumor cells, the specific experimental methods and results are as follows:

NCI-H157 cells were inoculated on a 24-well cell culture plate and subjected to gradient dosing treatment for time needed after cell adhesion. The cells were fixed at room temperature with a 4% solution of paraformaldehyde. The cells were washed with PBS buffer several times. An appropriate amount of human recombinant PD-1Fc protein was added to the fixed cells and incubated for 1 h, and the cells were washed with PBS buffer several times. Anti-human IgG (Alexa Fluor 488) fluorescent secondary antibody was added and incubated for 30 min. After nuclear staining, a glycerol sealing layer was applied. Green fluorescence signals were observed under a fluorescence microscope and photographed for statistics, or the fluorescence intensity was measured by flow cytometry.

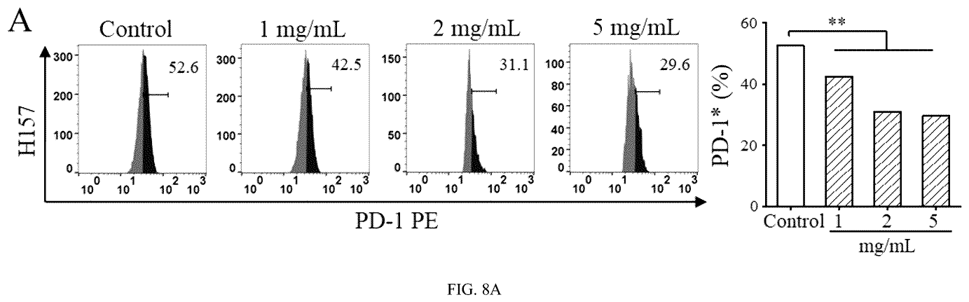

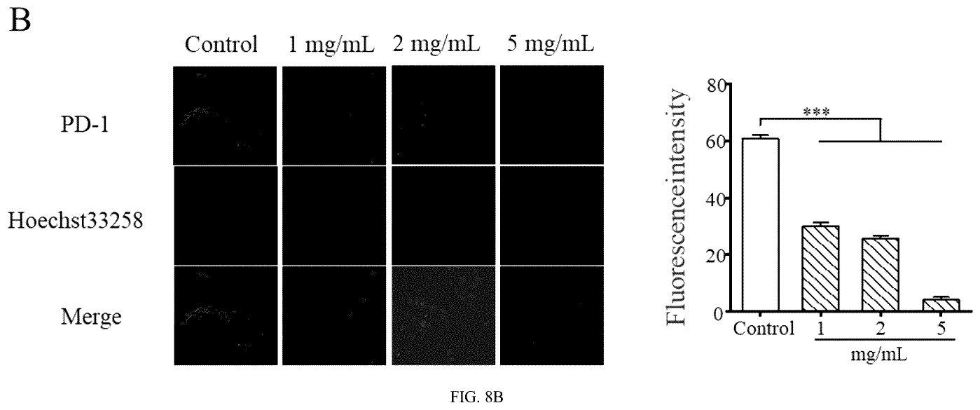

Both flow (see FIG. 8A) and confocal (see FIG. 8B) experimental results showed that the PD-1 binding to PD-L1 in NCI-H157 cells decreased after 24 h of ME-2 treatment, indicating that ME-2 could block the interaction between PD-L1 and PD-1 in a dose-dependent manner. It was suggested that ME-2 inhibited the biological function of immune checkpoints PD-1/PD-L1 by blocking the interaction of PD-1/PD-L1, and blocked the immune escape of tumor cells.

### (8) Killing Effect of Jurkat T Cells on Lung Cancer Cells Mediated by Auricularia Auricula Monomeric Polysaccharide ME-2

In order to further evaluate the anti-tumor effect of ME-2 in the experiment of co-culture of tumor cells and T cells, H157 cells were inoculated into a 24-well culture plate, subjected to gradient dosing treatment for time needed, and co-cultured with stimulated Jurkat T cells for 24 h. The tumor survival was observed under a microscope after 4% polyformaldehyde fixation and crystal violet staining. The specific manifestation was the difference in the depth of staining in the different dosages and concentrations of ME-2.

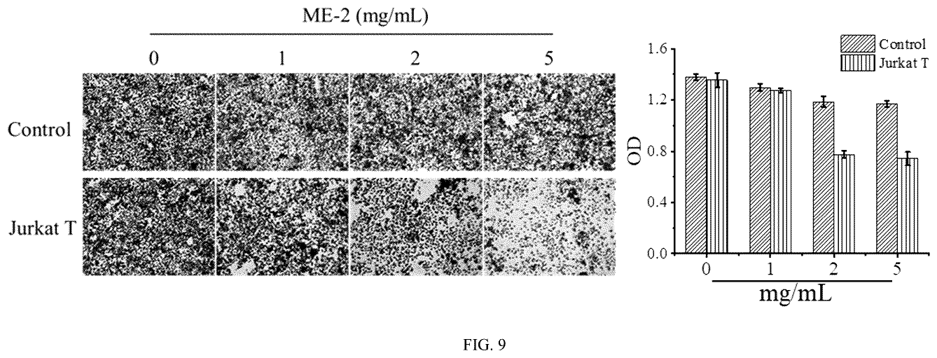

The experimental results are as follows: NCI-H157 cells treated with ME-2 were more sensitive to the killing of Jurkat T cells (see FIG. 9), which further proved that ME-2 mediated Jurkat T cell infiltration to kill tumor cells and down-regulated the expression of PD-L1 on the cell surface; the binding of tumor cells PD-L1 and Jurkat T cells PD-1 was inhibited in a concentration-dependent manner, and the immune escape of tumor cells was blocked.

(9) Application of Auricularia auricula Monomeric Polysaccharide ME-2 as PD-L1 Inhibitor

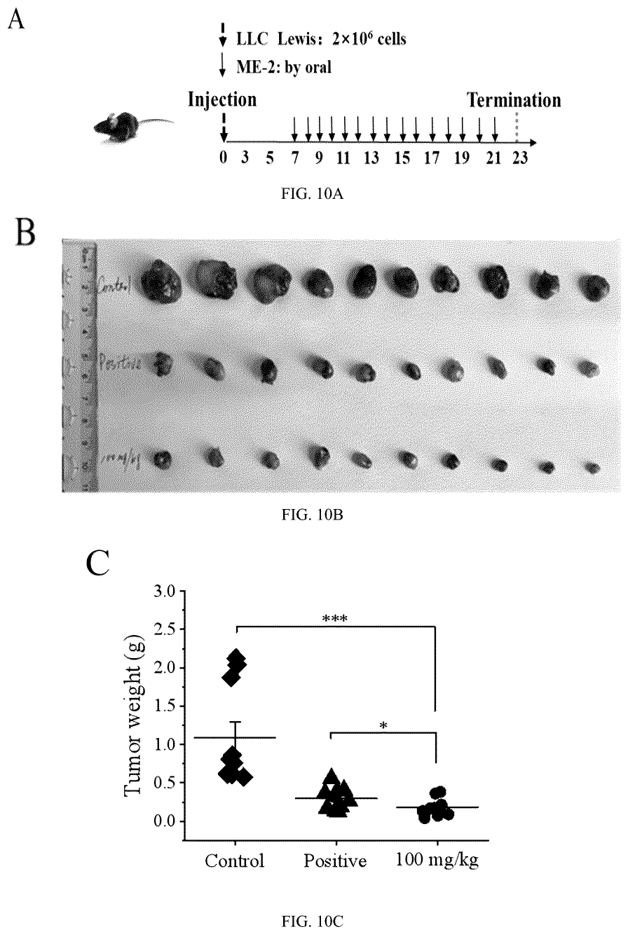

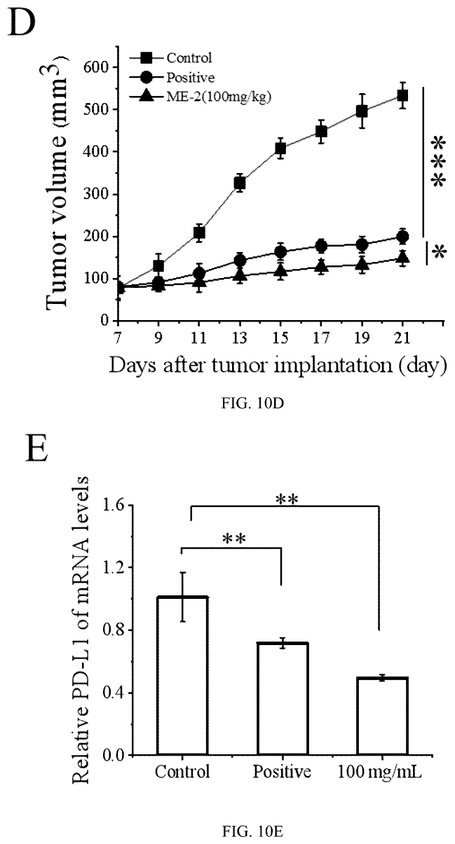

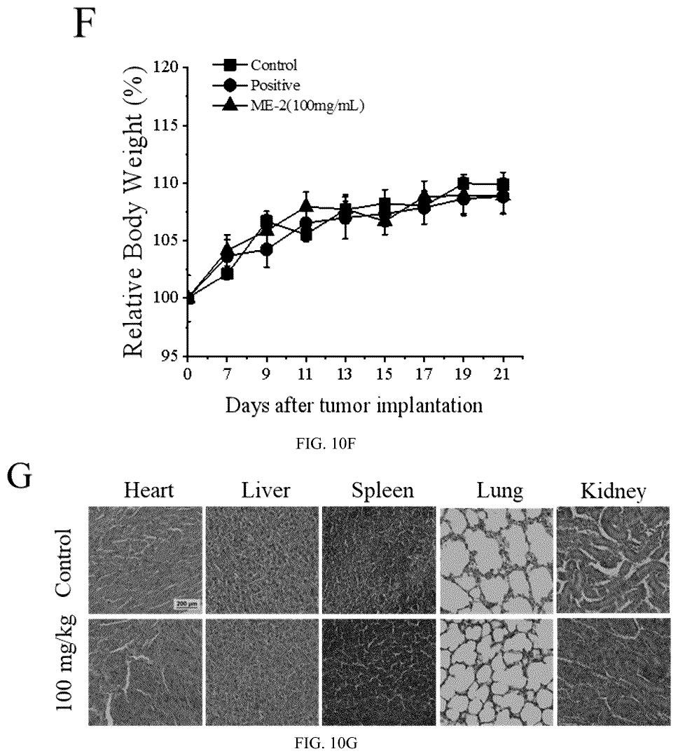

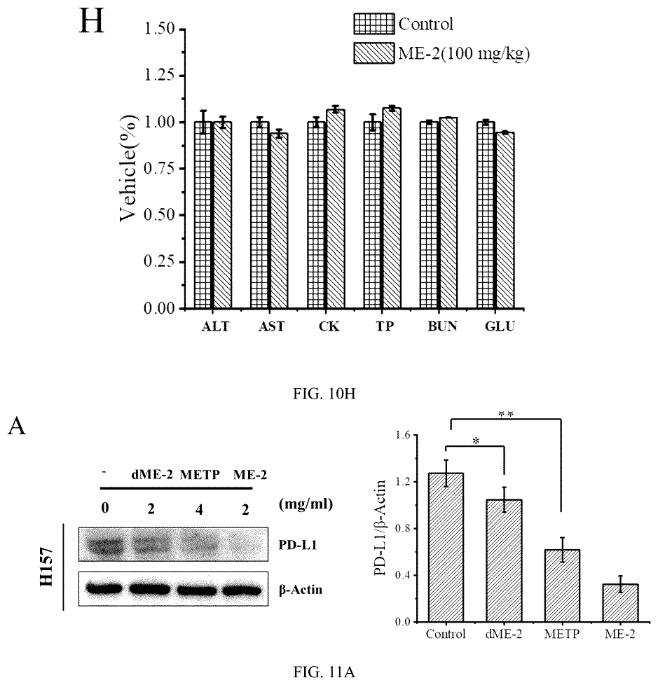

In order to explore the anti-tumor effect of ME-2 in vivo, a mouse lung cancer transplanted tumor model was established (see FIG. 10A). Experimental results showed that tumor growth was effectively inhibited in the model mice treated with ME-2 measured at 100 mg/kg (see FIG. 10B). Specifically, with the accumulation of administration time, the tumor volume of mice in the ME-2 administration group increased slowly overall, and ME-2 showed good anti-tumor activity (see FIGS. 10C, 10D). In order to further verify that Auricularia auricula monomeric polysaccharide ME-2 can be used as a PD-L1 inhibitor, it was confirmed by qPCR that ME-2 can significantly reduce the expression of PD-L1 mRNA level in mouse tumors in vitro, and the expression results are shown in FIG. 10E. It was found that there was no significant difference in body weight among all groups (see FIG. 10F). The results of hematoxylin-eosin (HE) staining of the vital organs of mice in 100 mg/kg experimental group showed that ME-2 did not cause organic changes in the organs of mice (see FIG. 10G), and all serum indexes were consistent with those of the control group (see FIG. 10H), indicating that ME-2 had no obvious toxic and side effects on mice at the dosage used.

### Experiment Example 2 the Experiment of Auricularia auricula Total Polysaccharide METP as PD-L1 Inhibitor

In order to explore whether Auricularia auricula total polysaccharide METP (prepared by Example 1) has medicinal value as a PD-L1 inhibitor, this experiment was verified from both in vitro and in vivo experiments.

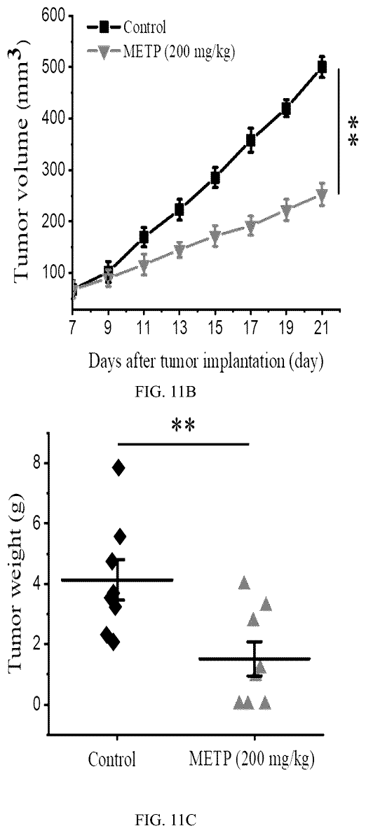

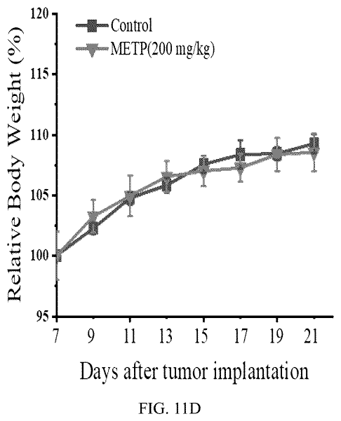

A Western blot was used to detect the effects of Auricularia auricula total polysaccharide METP and Auricularia auricula deacetylated monomeric polysaccharide dME-2 on the expression of PD-L1 in H157 cells, and Auricularia auricula monomeric polysaccharide ME-2 was used as a positive control. The experimental procedure was described in Example 1. The results showed that Auricularia auricula total polysaccharide METP could significantly down-regulate the expression of PD-L1 protein in H157 cells, while the down-regulation effect of Auricularia auricula deacetylated monomeric polysaccharide dME-2 on PD-L1 protein in H157 cells was significantly weaker than that of ME-2, indicating that the acetyl active group is the key active group for ME-2 to act as a PD-L1 inhibitor. Detailed results are shown in FIG. 11A.

On this basis, the Auricularia auricula total polysaccharide was used as a PD-L1 inhibitor in the tumor-bearing mouse model in this experiment. The LLC lung cancer transplanted tumor model of C57BL/6 tumor-bearing mice was established, and the specific procedure was the same as that of Example 1. The results showed that Auricularia auricula total polysaccharide METP (200 mg/kg) could effectively inhibit the proliferation of tumors in mice and showed good anti-tumor activity. There was no significant difference in mouse body weight among all groups, which confirmed the biosafety of Auricularia auricula total polysaccharide METP (see FIGS. 11B-11D). In conclusion, the Auricularia auricula total polysaccharide METP can act as a PD-L1 inhibitor, blocking the immune escape mechanism of PD-L1/PD-1 by down-regulating the expression of PD-L1 in tumor cells, and playing an anti-tumor role.

What is claimed is:

1. A preparation method of a programmed cell death ligand 1 (PD-L1) inhibitor, comprising using an Auricularia auricula total polysaccharide, wherein a content of acetyl active groups in the Auricularia auricula total polysaccharide is more than 10%.

2. The preparation method according to claim 1, wherein a preparation method of the Auricularia auricula total polysaccharide comprises:
(1) extracting Auricularia auricula with water as an extraction solvent to obtain an Auricularia auricula water extract; and
(2) removing an impurity in the Auricularia auricula water extract to obtain the Auricularia auricula total polysaccharide containing the acetyl active groups.

3. The preparation method according to claim 2, wherein in the step (1) of the preparation method of the Auricularia auricula total polysaccharide, the water is used as the extraction solvent to extract the Auricularia auricula water extract from the Auricularia auricula by a boiling water extraction method; the boiling water extraction method comprises a decocting method or a reflux extraction method; the impurity in the step (2) of the preparation method of the Auricularia auricula total polysaccharide comprises a small-molecule compound, an oligosaccharide, or a soluble salt; and a method for removing the impurity in the Auricularia auricula water extract comprises an interception treatment of the Auricularia auricula water extract by using an ultrafiltration chromatographic column, wherein an interception treatment range of the ultrafiltration chromatographic column is selected from 3K, 5K, 10K, or 50K.

4. The preparation method according to claim 1, wherein a molecular weight distribution of the Auricularia auricula total polysaccharide comprises 1737 KDa, 308.5 KDa, and 145.4 KDa; according to a molar percentage of a monosaccharide composition, xylose:glucuronic acid:galactose:glucose:mannose=25:7:2:3:63; the content of the acetyl active groups is 2.47%.

5. A preparation method of a PD-L1 inhibitor, comprising using a monomeric polysaccharide isolated from Auricularia auricula, wherein a content of acetyl active groups of the monomeric polysaccharide is 18.0%.

6. The preparation method according to claim 5, wherein the monomeric polysaccharide is a high-acetyl glucuronoxylogalactoglucomannan, and the high-acetyl glucuronoxylogalactoglucomannan is composed of xylose, glucuronic acid, galactose, glucose, and mannose, wherein a molar ratio of the xylose:the glucuronic acid:the galactose:the glucose:the mannose is 3:4:1:1:11;
a sugar residue linking composition of the monomeric polysaccharide is as follows: →3)-Manp-(1→, →2,3)-Manp-(1→, →2,3,6)-Manp-(1→, →3,6)-Manp-(1→, Manp-(1→, Glcp-(1→, GlcAp-(1→, Xylp-(1→, and Galp-(1→; and the →3)-Manp-(1→, →2,3)-Manp-(1→, →2,3,6)-Manp-(1→, →3,6)-Manp-(1→, Manp-(1→, Glcp-(1→, GlcAp-(1→, Xylp-(1→, and Galp-(1→; a theoretical ratio was 3:1:3:3:1:1:4:3:1.

7. The preparation method according to claim 6, wherein a chemical fine structure repeating unit of the monomeric polysaccharide is shown as follows:

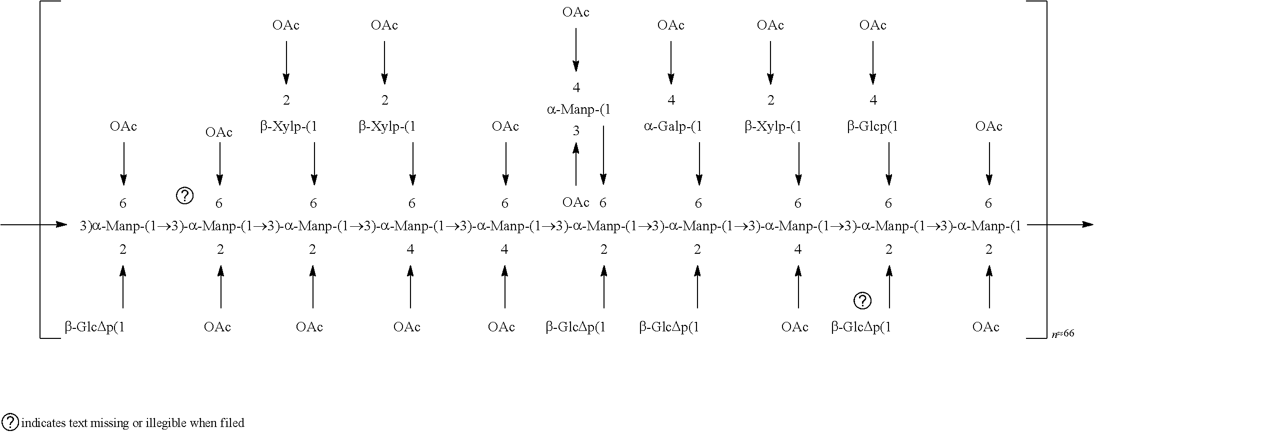

8. A pharmaceutical composition for inhibiting PD-L1 or treating a tumor, comprising an effective amount of an Auricularia auricula total polysaccharide or an Auricularia auricula monomeric polysaccharide and an acceptable excipient or carrier in a pharmaceutical preparation; wherein the Auricularia auricula total polysaccharide or the Auricularia auricula monomeric polysaccharide comprises acetyl active groups; the tumor comprises non-small cell lung cancer, osteosarcoma, colon cancer, liver cancer, and breast cancer.

9. The pharmaceutical composition according to claim 8, wherein a preparation method of the Auricularia auricula total polysaccharide comprises:
(1) extracting Auricularia auricula with water as an extraction solvent to obtain an Auricularia auricula water extract; and
(2) removing an impurity in the Auricularia auricula water extract to obtain the Auricularia auricula total polysaccharide containing the acetyl active groups;
the Auricularia auricula monomeric polysaccharide is a high-acetyl glucuronoxylogalactoglucomannan, the high-acetyl glucuronoxylogalactoglucomannan is composed of xylose, glucuronic acid, galactose, glucose, and mannose, wherein a molar ratio of the xylose:the glucuronic acid:the galactose:the glucose:the mannose is 3:4:1:1:11; a sugar residue linking composition of the Auricularia auricula monomeric polysaccharide is as follows: →3)-Manp-(1→, →2,3)-Manp-(1→, →2,3,6)-Manp-(1→, →3,6)-Manp-(1→, Manp-(1→, Glcp-(1→, GlcAp-(1→, Xylp-(1→, and Galp-(1→; and the →3)-Manp-(1→, →2,3)-Manp-(1→, →2,3,6)-Manp-(1→, →3,6)-Manp-(1→, Manp-(1→, Glcp-(1→, GlcAp-(1→, Xylp-(1→, and Galp-(1→; a theoretical ratio was 3:1:3:3:1:1:4:3:1.

10. The pharmaceutical composition according to claim 8, wherein the pharmaceutical composition is prepared into a conventional pharmaceutical preparation according to a conventional method in a field of pharmaceutical preparations; a dosage form of the conventional pharmaceutical preparation is a solid preparation, a semi-solid preparation, or a liquid preparation.

11. The preparation method according to claim 1, wherein the content of the acetyl active groups in the Auricularia auricula total polysaccharide is 15-30%.

12. The pharmaceutical composition according to claim 10, wherein the dosage form of the conventional pharmaceutical preparation is a freeze-dried powder, a tablet, a capsule, a soft capsule, a granule, a pill, an oral liquid, a dry suspension, a dropping pill, a dry extract, an injection, or an infusion.
